(19) United States
(12) Patent Application Publication
(10) Pub. No.: US 2024/0032779 A1
(43) Pub. Date: Feb. 1, 2024

(54) ENDOSCOPE AND ENDOSCOPE TRANSPORTATION UNIT

(71) Applicant: OLYMPUS MEDICAL SYSTEMS CORP., Tokyo (JP)

(72) Inventor: Seiji IWASAKI, Tokyo (JP)

(73) Assignee: OLYMPUS MEDICAL SYSTEMS CORP., Tokyo (JP)

(21) Appl. No.: 18/227,388

(22) Filed: Jul. 28, 2023

Related U.S. Application Data
(60) Provisional application No. 63/393,982, filed on Aug. 1, 2022.

(51) Int. Cl. A61B 1/00; A61B 50/30

(52) U.S. Cl. CPC A61B 1/00128; A61B 50/30

(57) ABSTRACT

An endoscope includes a tube including a cable, an operation section connected to the tube, and a connector unit that connects the tube and the operation section. The operation section inserts the cable. The connector unit is movable between a first state and a second state to change a separation distance between a distal end of the tube and the operation section.

### RELATED APPLICATION DATA

This application is based on and claims priority under 37 U.S.C. § 119 to U.S. Provisional Application No. 63/393,982 filed on Aug. 1, 2022, the entire contents of which are incorporated herein by reference.

### BACKGROUND

The present disclosure relates to an endoscope in which a cable is extended from an operation section, and an endoscope transportation unit.

In medical fields and industrial fields, endoscopes have been widely used for various tests. An endoscope includes an insertion section that is inserted into a subject.

In the endoscope, a universal cord, which is a composite cable, is extended from an operation section. A scope connector connected to external equipment such as a video processor or a light source apparatus is provided at an end portion of the universal cord.

For example, Japanese Patent Application Laid-Open Publication No. 2008-228953 discloses a technique of an endoscope in which a rotary connector is provided in a connecting portion of an operation section and a universal cord to prevent twists from being accumulated in the universal cord.

### SUMMARY

An endoscope according to an aspect of the present disclosure includes a tube including a cable, an operation section connected to the tube, and a connector unit that connects the tube and the operation section. The operation section inserts the cable. The connector unit is movable between a first state and a second state to change a separation distance between a distal end of the tube and the operation section.

### BRIEF DESCRIPTION OF THE DRAWINGS

FIG. 1 is a diagram showing a configuration of an endoscope.

FIG. 2 is a diagram showing a configuration of the endoscope in a state in which a universal cord comes close to an operation section.

FIG. 3 is a diagram showing a configuration of a cable connector.

FIG. 4 is a diagram showing a state in which the cable connector is turned and the universal cord comes close to the operation section.

FIG. 5 is a perspective view showing a configuration of a hollow joint member.

FIG. 6 is a diagram showing an endoscope transportation unit in which the endoscope is packed in a box.

FIG. 7 is a perspective view showing a configuration of a stopper mechanism in a first modification.

FIG. 8 is a perspective view showing a configuration of two base rings in the first modification.

FIG. 9 is a perspective view showing a configuration of a stopper mechanism in a second modification.

FIG. 10 is a perspective view showing a configuration of two base rings in the second modification.

FIG. 11 is a diagram showing a configuration of a cable connector of a ball joint mechanism in a third modification.

FIG. 12 is a diagram showing a state in which the cable connector of the ball joint mechanism in the third modification is turned and the universal cord comes close to the operation section.

FIG. 13 is a diagram showing a configuration of a cable connector formed from a tube body of an elastic member in a fourth modification.

FIG. 14 is a diagram showing a state in which a cable connector formed from a soft tube body in the fourth modification is turned and the universal cord comes close to the operation section.

FIG. 15 is a diagram showing an endoscope transportation unit in which an endoscope including a cable connector formed from a tube body of an elastic member in the fourth modification is packed in a box.

### DETAILED DESCRIPTION

In the endoscope of the related art, a hard member for bend prevention is provided in the connecting portion of the operation section and the universal cord in order to prevent a kink and the like of the universal cord. An angle of the hard member with respect to the operation section and a shape of the hard member are set to form the endoscope in a shape which can be easily grasped by a user.

In the endoscope, the hard member provided in the connecting portion of the operation section and the universal cord extends while having an angle with respect to a longitudinal direction of the operation section. Therefore, the endoscope cannot be formed in a compact state because of the hard member extending from the operation section. Consequently, the endoscope has a problem that the endoscope is bulky and is poor in portability at a transportation time or the like.

Therefore, an object of the present disclosure is to realize an endoscope that can be formed in a compact state in order to improve portability at a transportation time, and an endoscope transportation unit.

An endoscope functioning as insertion equipment in the present embodiment is explained as an example. Note that, in the following explanation, drawings based on respective embodiments are schematic and relations between thicknesses and widths of respective portions, ratios of the thicknesses of the respective portions, and the like are different from real ones. Portions different in relations and ratios of dimensions from one another are sometimes included among the drawings.

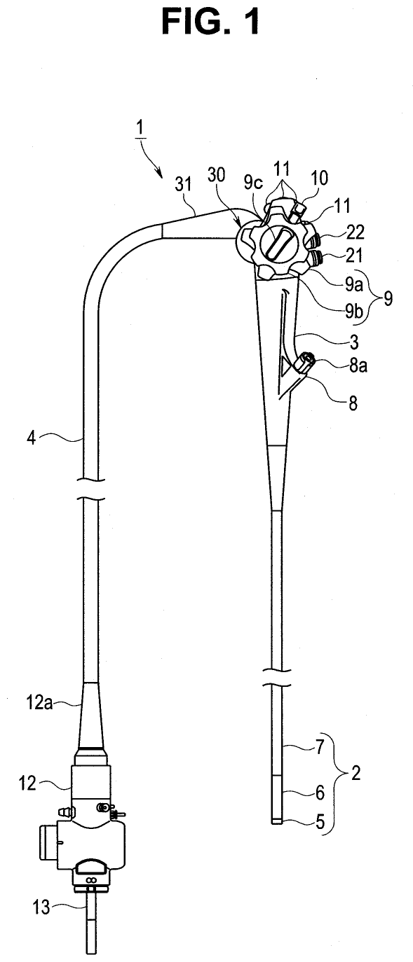

First, a schematic configuration of an endoscope 1 is explained. The endoscope 1 includes, as shown in FIG. 1, an endoscope main body portion including an insertion section 2, an operation section 3, and a universal cord (tube) 4.

The insertion section 2 is a long member insertable into an inside of a subject. The operation section 3 is consecutively connected to a proximal end of the insertion section 2. The universal cord 4, which is a scope cable, is a composite cable extending from a side portion of the operation section 3.

The insertion section 2 includes a distal end portion 5, a bending section 6, a flexible tube section 7, which is a flexible tube. The distal end portion 5 is disposed at a distal end of the insertion section 2. The bending section 6 is actively bendable. The bending section 6 is consecutively connected to a proximal end side of the distal end portion 5. The flexible tube section 7 is consecutively connected to the proximal end side of the bending section 6. The flexible tube section 7 is connected to extend from a distal end of the operation section 3.

The operation section 3 includes a treatment instrument insertion port 8, an angle knob 9, a UD angle fixing lever 10, remote switches 11, a gas/liquid feeding button 21, and a suction button 22.

The treatment instrument insertion port 8 is an opening section communicating with a treatment instrument insertion channel (not shown), which is an endoscope conduit. A forceps plug 8a, which is a forceps pipe sleeve, is provided in the treatment instrument insertion port 8.

The angle knob 9 is an operation handle for operating a bend of the bending section 6. The angle knob 9 includes a UD angle knob 9a and an RL angle knob 9b.

The UD angle knob 9a is an operation handle for operating a bend in an up-down direction of the bending section 6. The RL angle knob 9b is an operation handle for operating a bend in a left-right direction of the bending section 6. Note that an RL angle fixing knob 9c superimposed on the RL angle knob 9b is disposed.

Four remote switches 11 are provided. The four remote switches 11 are operation switches of a video system respectively allocated to perform image stop, recording, photometry switching, image enlargement, and the like.

The gas/liquid feeding button 21 is an operation button for controlling a fluid feeding operation from a fluid feeding section (not shown) provided at the distal end portion 5. It is possible to adopt, for example, a structure in which gas feeding can be performed by a small hole in a center of the gas/liquid feeding button 21 being closed by a finger. It is possible to adopt, for example, a structure in which liquid feeding can be performed by the gas/liquid feeding button 21 being pushed in.

The suction button 22 is an operation button for controlling an inside of the treatment instrument insertion channel (not shown) to a negative pressure. It is possible to suction and remove mucus or the like adhering to the distal end portion 5 from an opening section (not shown) of the treatment instrument insertion channel by the suction button 22 being pushed in.

A cable connector 30, from which an arm 31 also functioning as cable bend prevention is extended, is disposed in the connecting portion of the universal cord 4 and the operation section 3. The cable connector 30 has a structure that is turnable with respect to the operation section 3. Note that a detailed configuration of the cable connector 30 is explained below.

A scope connector 12 of the endoscope main body portion is disposed on the proximal end side of the universal cord 4. The scope connector 12 includes a bend preventing portion 12a in a connecting portion to the universal cord 4.

The scope connector 12 is connected to a video processor or the like (not shown) of an external apparatus including a light source apparatus. Besides an electric contact and the like, a light guide connector 13 is extended from the scope connector 12.

The video processor, to which the scope connector 12 is connected, transmits light emitted from an internal light source to a not-shown light guide bundle inserted through the universal cord 4, the operation section 3, and the insertion section 2. The light transmitted to the light guide bundle is emitted as illumination light from an illumination window (not shown) provided at the distal end portion 5.

Note that the distal end portion 5 includes an observation window (not shown). The distal end portion 5 includes an image sensor (not shown) that photoelectrically changes photographing light made incident from the observation window. An image pickup cable extending from the image sensor is inserted through the insertion section 2, the operation section 3, and the universal cord 4 and disposed to the scope connector 12.

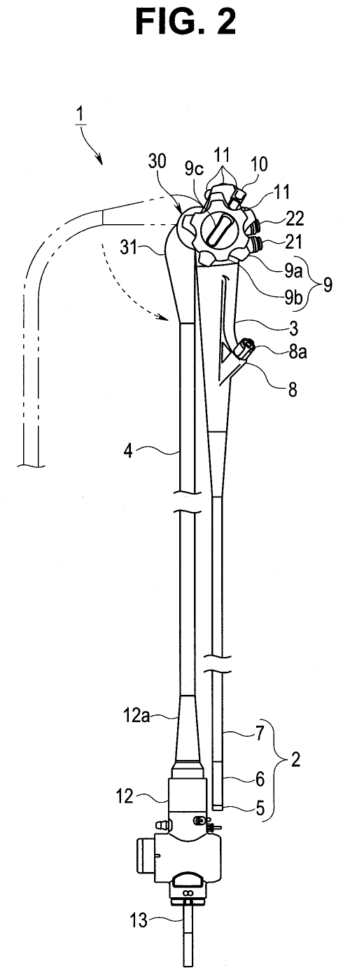

Subsequently, a configuration of the cable connector 30 of the endoscope 1 is explained in detail below. The cable connector 30 in the present embodiment is a mechanism that is deformable (movable) into a used state, which is a first state in which, as shown in FIG. 1, the arm 31 is turned and the universal cord 4 connected to the arm 31 separates from the operation section 3, and an unused state, which is a second state in which, as shown in FIG. 2, the universal cord 4 comes close to the operation section 3. Note that a state in which the endoscope 1 is not used is, for example, a state at a transportation time of the endoscope 1 and a time for storing the endoscope 1 in a hospital.

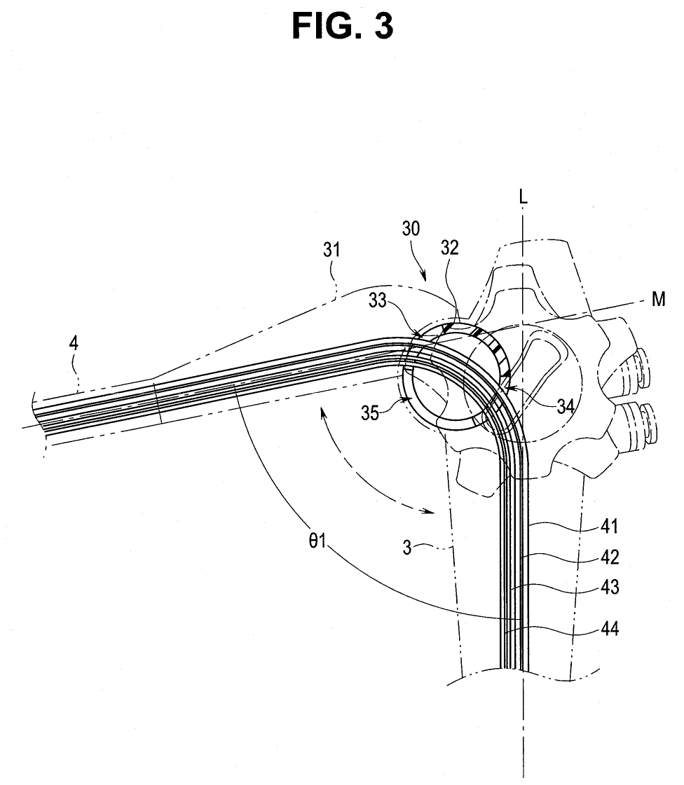

The cable connector 30 is provided on the proximal end side of the operation section 3 that is a paper surface upward direction when viewed facing the drawing. Inside the cable connector 30, as shown in FIG. 3 to FIG. 5, a hollow joint member (first body) 32, which is a substantially tubular turning member, is provided. The hollow joint member 32 is a tube body extending in an axis (connector axis) X direction.

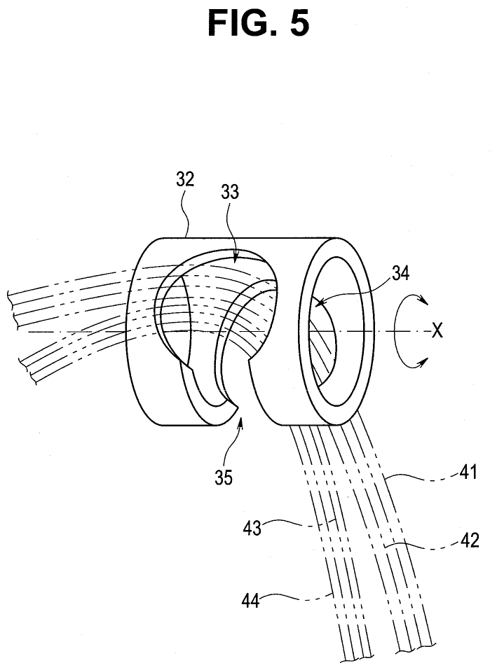

In the hollow joint member 32, as shown in FIG. 5, a first cutout (first cutout region) 33 and a second cutout (second cutout region) 34, which are circular two hole sections, are formed in a side circumferential portion of the tube body. The first cutout 33 and the second cutout 34 are formed to have a predetermined distance around an axis (a rotation axis) X of the hollow joint member 32. That is, the first cutout 33 and the second cutout 34 are formed in facing positions in a side circumference of the hollow joint member 32. The connector unit 30 rotates around the connector axis X. The cable 41, 42, 43 or 44 extends in a first direction, and the connector axis X is oriented at an angle to the first direction. The connector unit 30 can include the first body 32, the first cutout region 33 formed in the first body 32, and the second cutout region 34 formed in the first body 32. The first body 32 can have tubular, oval or circular shape that is or is not the same as the second body 36. The second body 36 can have tubular, oval or circular shape. The connector unit can include the third cutout region 35 formed in the first body 32 and connecting the first cutout region 33 and the second cutout region 34. A width of the third cutout region 35 in a direction of the connector axis X is smaller than a width of the first cutout region 33 in the direction of the connector axis X.

Further, in the hollow joint member 32, a groove-like third cutout (third cutout region) 35 connecting the first cutout 33 and the second cutout 34 is formed. In the third cutout 35, a width in a rotation axis (axis X) direction of the hollow joint member 32 is set smaller than respective diameters of the first cutout 33 and the second cutout 34.

The hollow joint member 32 is held turnably around the axis X in an exterior of the operation section 3. The hollow joint member 32 is connected to the arm 31. That is, the arm 31 is also turnable together with the hollow joint member 32 with respect to the operation section 3. The arm 31 is also a hollow member. The universal cord 4 is connected to an end portion of the arm 31.

Note that the arm 31 includes two stoppers (not shown) that abut the operation section 3. That is, the cable connector 30 can maintain, with one stopper of the arm 31 that abuts the operation section 3, a state in which turning of the arm 31 is stopped in a predetermined position where the endoscope 1 is in the used state, which is the first state in which the universal cord 4 separates from the operation section 3.

The cable connector 30 can maintain, with the other stopper of the arm 31 that abuts the operation section 3, a state in which the turning is stopped in a predetermined position where the endoscope 1 is in the unused state, which is the second state in which the universal cord 4 comes close to the operation section 3.

Note that at least one of long members including an electric cable 41 such as the image pickup cable disposed in the insertion section 2 and the operation section 3, a light guide 42 of an optical fiber, a gas feeding tube 43, and a liquid feeding tube 44 is inserted through the universal cord 4 passing through the cable connector 30. The tube 4 includes the cable 41, 42, 43 or 44. The operation section 3 is connected to the tube, and the connector unit 30 connects the tube 4 and the operation section 3. The operation section 3 inserts the cable 4. The connector unit 30 is movable between the first state and the second state to change a separation distance between a distal end of the tube 4 and the operation section 3.

The long members including the electric cable 41, the light guide 42 of the optical fiber, the gas feeding tube 43, and the liquid feeding tube 44 are inserted through the first cutout 33, the second cutout 34, and third cutout 35 of the hollow joint member 32.

In the endoscope 1 configured as explained above, the cable connector 30 of a scope cable turning mechanism that turns in an attaching portion of the universal cord 4, which is the scope cable extended from the operation section 3, is provided. That is, in the endoscope 1, the universal cord 4 is turnably coupled to the operation section 3 by the cable connector 30.

Therefore, in the endoscope 1, by turning the cable connector 30 with respect to the operation section 3, which is a first portion, it is possible to bring the universal cord 4, which is a second portion, close to the operation section 3 side and compactly fold the universal cord 4 extended in an axis M direction crossing a longitudinal axis L of the operation section 3.

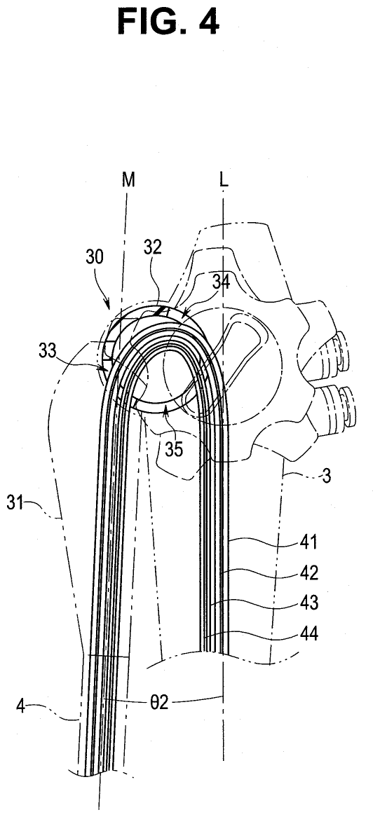

That is, since the endoscope 1 includes the cable connector 30, it is possible to change an extending direction of the axis (longitudinal axis) M of the universal cord 4 crossing the longitudinal axis L of the operation section 3 while separating at a predetermined angle (first angle) 01 in a used state shown in FIG. 3 to a predetermined angle (second angle) 02 at which the universal cord 4 comes close to the operation section 3 in an unused state shown in FIG. 4.

In the first state, the tube 4 is located at the first angle θ1 with respect to the longitudinal axis of the operation section 3. In the second state, the tube 4 is located at the second angle θ2 with respect to the longitudinal axis of the operation section 3. The first angle θ1 is larger than the second angle θ2. In the first state, the separation distance is a first separation distance. In the second state, the separation distance is a second separation distance. The first separate distance is larger than the second separation distance.

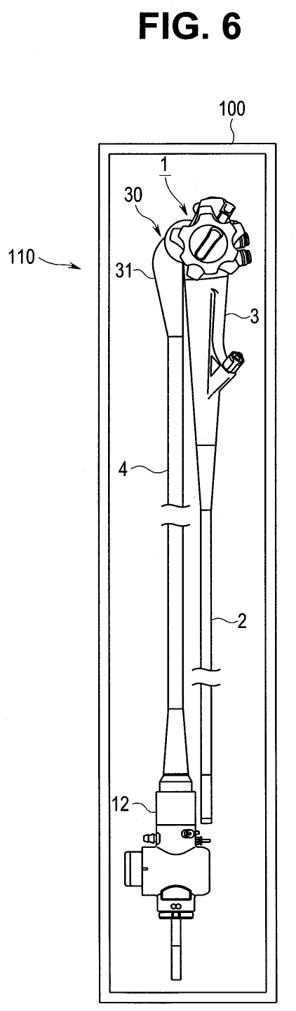

Consequently, the endoscope 1 can be packed in, for example, an elongated box 100, which is an endoscope case, of an endoscope transportation unit 110 shown in FIG. 6. Since the packing of the endoscope 1 can be made compact by the elongated box 100 in this way, the endoscope 1 is not bulky and transportation cost can be reduced. Further, for example, when a manufacturer collects the used endoscope 1, it is unnecessary to disassemble the endoscope 1. It is possible to reduce expenses for repair cost. The endoscope transportation unit 110 can include the endoscope 1, and the case 100 that stores the endoscope 1. When the endoscope 1 is in the second state, a width of the endoscope 1 is less than a width of the case 100. When the separation distance is a minimum, the separation distance is less than the width of the case.

As explained above, the endoscope 1 in the present embodiment can be deformed into a compact state in which the universal cord 4 comes close to the operation section 3. Therefore, it is possible to improve portability at a transportation time.

### (First Modification)

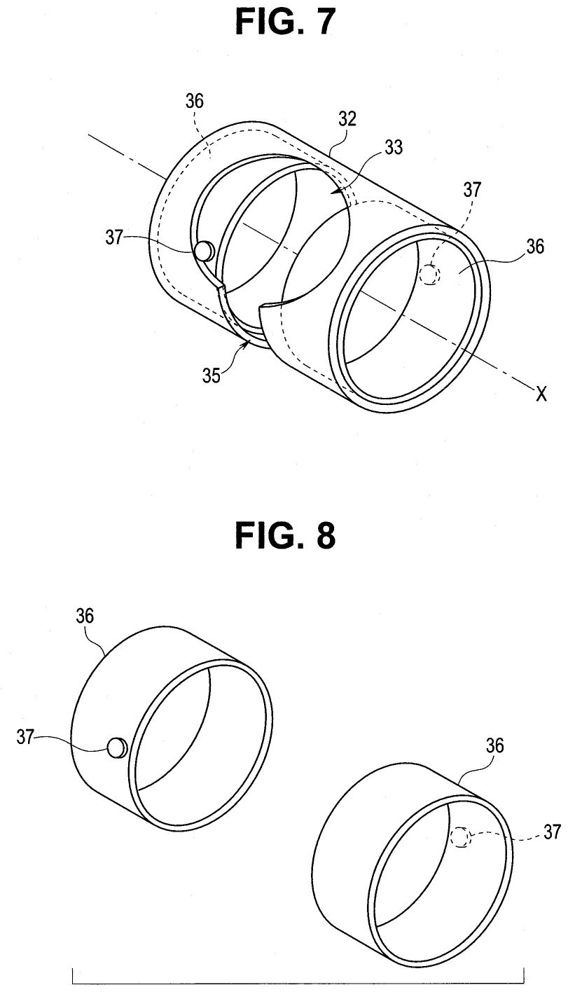

A stopper mechanism for restricting (defining) a movable range of the cable connector 30 may be a configuration in which, as shown in FIG. 7 and FIG. 8, base rings (second bodies) 36 are provided inside the hollow joint member (first body) 32 to restrict turning of the cable connector 30.

More specifically, two base rings 36 are provided to slide on an inner circumferential surface of the hollow joint member 32. Projections 37 are provided on respective outer circumferential surfaces of the two base rings 36.

One of the projections 37 of the base rings 36 comes into contact with an edge side portion of the first cutout 33 of the hollow joint member 32, the other comes into contact with an edge side portion of the second cutout 34, and the projections 37 abut with each other, whereby turning of the hollow joint member 32 is restricted.

Note that the two base rings 36 are engaged with both end sides of the hollow joint member 32. The two base rings 36 are fixed to the operation section 3 not to turn.

The two base rings 36 may be formed from resin and machine into one body including the respective projections 37 or may be formed from metal such as stainless steel and configured by press-fitting pins, which are the projections 37, into holes of rings.

Note that a coat of silicone resin may be applied to a part of the outer circumferential surfaces near the projections 37 of the base rings 36 to restrict the turning of the hollow joint member 32 making use of contact friction between the coat of the silicone resin and the inner circumferential surface of the hollow joint member 32. That is, the contact friction may be increased to restrict the turning.

In this way, with the stopper mechanism in which the two base rings 36 are provided inside the hollow joint member 32, it is possible to maintain a state in which the turning is stopped in the predetermined positions of the used state, which is the first state in which the universal cord 4 separates from the operation section 3, and the unused state, which is the second state in which the universal cord 4 comes close to the operation section 3.

The connector unit can include the first body 32, the first cutout region 33 formed in the first body 32, and the second cutout region 34 formed in the first body 32. The connector unit can include the third cutout region 35 formed in the first body 32, and the third cutout region 35 connects the first cutout region 33 and the second cutout region 34. A width of the third cutout region 35 in a direction of the connector axis X is smaller than a width of the first cutout region 33 in the direction of the connector axis X.

The connector unit can include the second body 36 inserted into the first body 32. The first body 32 can rotate relative to the second body 36. The second body 36 can include the projection 37 on the outer circumferential surface of the second body 36. The projection 37 restricts rotation of the first body 32 relative to the second body 36. The second body 36 can rotate relative to the first body 32. The projection 37 extends through the first cutout region 33. When the first body 32 rotates relative to the second body 36, the projection 37 contacts a periphery surface of the first cutout region 33 to restrict rotation of the first body 32 relative to the second body 36. The projection 37 can be formed of silicone resin.

### (Second Modification)

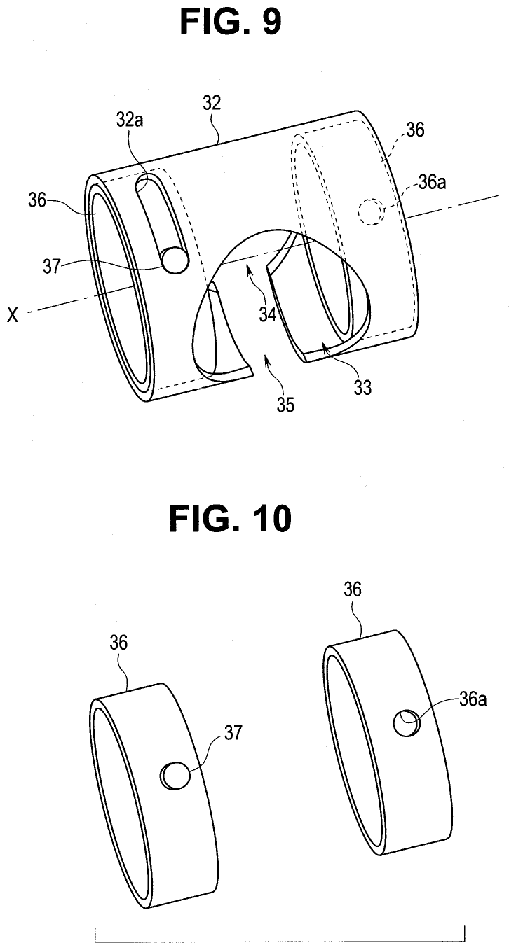

The stopper mechanism for restricting the movable range of the cable connector 30 may be a cam mechanism in which, as shown in FIG. 9, a cam groove (groove) 32a, which is a slit, is formed in the hollow joint member 32 such that the projection 37 of the base ring 36 engages in the cam groove 32a.

That is, the projection 37 is disposed in the cam groove 32a of the hollow joint member 32. The projection 37 comes into contact with and abuts an end portion of the cam groove 32a, whereby the turning of the hollow joint member 32 is restricted.

Note that the base ring 36 is formed of metal such as stainless steel and a pin, which is the projection 37, is press-fit and provided in a hole 36a as shown in FIG. 10. In one base ring 36, the projection 37 is not provided in the hole 36a. In order to reduce the number of components, the projection 37 is provided in one of the two base rings 36 and the projection 37 is not provided in the other.

Further, as in the first modification, a coat of silicone resin may be applied to a part of the outer circumferential surfaces near the projections 37 of the base rings 36 to restrict the turning of the hollow joint member 32 making use of contact friction between the coat of the silicone resin and the inner circumferential surface of the hollow joint member 32.

The connector unit can include the first body 32, the first cutout region 33 formed in the first body 32, and the second cutout region 34 formed in the first body 32. The connector unit can include the third cutout region 35 formed in the first body 32, and the third cutout region 35 connects the first cutout region 33 and the second cutout region 34. A width of the third cutout region 35 in a direction of the connector axis X is smaller than a width of the first cutout region 33 in the direction of the connector axis X. The connector unit can include the second body 36 inserted into the first body 32. The first body 32 can rotate relative to the second body 36. The second body 36 includes the projection 37 on the outer circumferential surface of the second body 36. The projection 37 restricts rotation of the first body 32 relative to the second body 36. The second body 36 can rotate relative to the first body 32. The first body 32 can include the groove 32a formed in the first body 32. The projection 37 extends through the groove 32a. When the first body 32 rotates relative to the second body 36, the projection 37 contacts a periphery surface of the groove 32a to restrict rotation of the second body 36 relative to the first body 32.

### (Third Modification)

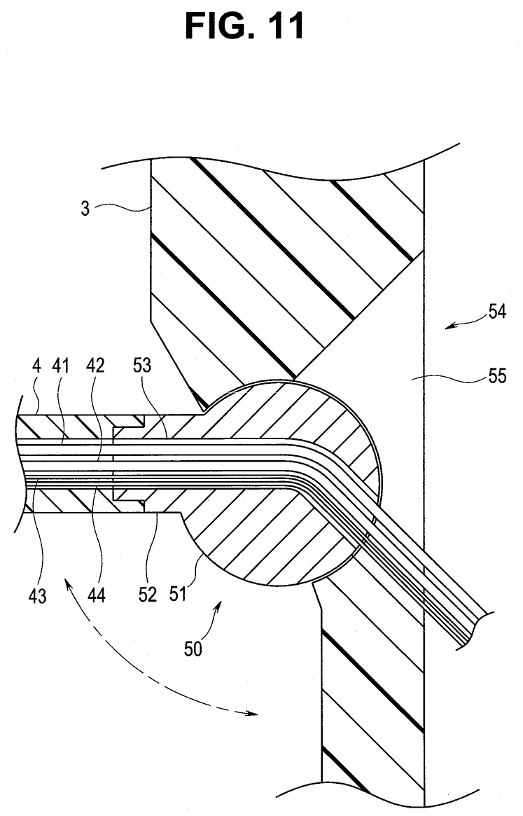

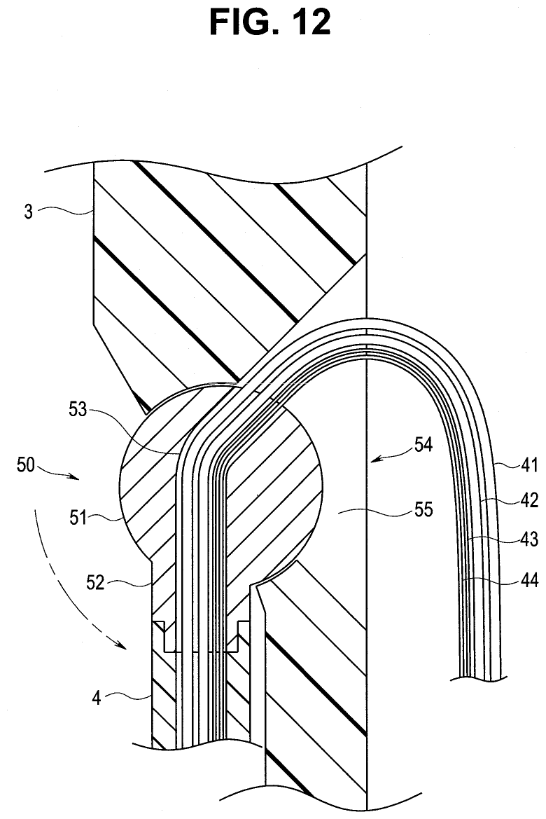

As shown in FIG. 11 and FIG. 12, in the endoscope 1, a configuration of a cable connector 50 may be a ball joint mechanism. The cable connector 50 includes a ball body (ball joint) 51 of a spherical member and a columnar ball stud 52 extending from the ball body. Note that the universal cord 4 is connected to the ball stud 52.

The ball body 51 engages in a socket 54 formed in the operation section 3 and is held to turn.

A first through-hole 53 is formed in the ball body 51 and the ball stud 52. The through-hole 53 is formed in a dogleg shape having a predetermined angle in the ball body 51. In the socket 54, a second through-hole 55 is formed on an inner side of the operation section 3.

The connector unit rotates around the connector axis. At least one of the cable 41, 42, 43 and 44 extends in the first direction, and the connector axis X is oriented at an angle to the first direction. The connector unit includes the ball joint 51. The ball joint 51 includes the first through-hole 53, and at least one of the cable 41, 42, 43 and 44 is inserted through the first through-hole. The operation section 3 includes the second through-hole 55. At least one of the cable 41, 42, 43 and 44 is inserted through the second through-hole 55. A surface of the ball joint 51 engages with a surface of the second through-hole 55 so that the ball joint 51 rotates relative to the second through-hole 55 when the connector unit moves between the first state and the second state.

The long members including the electric cable 41, the light guide 42 of the optical fiber, the gas feeding tube 43, and the liquid feeding tube 44 are inserted through the first through-hole 53 and the second through-hole 55.

In this way, in the cable connector 50 of the ball joint mechanism as well, the ball body 51 held by the socket 54 of the operation section 3 turns, whereby the universal cord 4 extended from the operation section 3 can be turnably coupled.

Consequently, in the endoscope 1, by turning the cable connector 50 with respect to the operation section 3, which is the first portion, it is possible to bring the universal cord 4, which is the second portion, close to the operation section 3 side and compactly fold the universal cord 4 extended in the axis M direction crossing the longitudinal axis L of the operation section 3.

As a result, since the endoscope 1 in this modification can also be deformed into a compact state in which the universal cord 4 comes close to the operation section 3, it is possible to improve portability at a transportation time.

### (Fourth Modification)

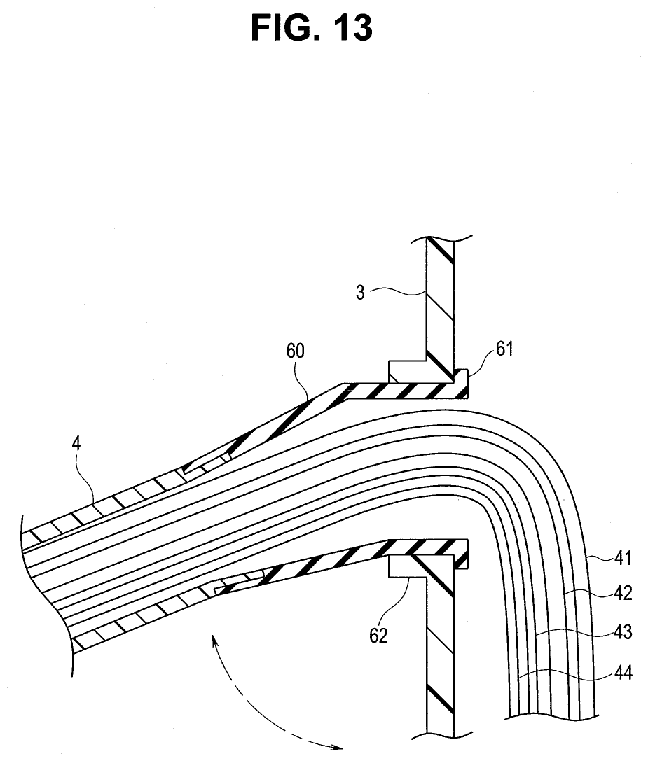

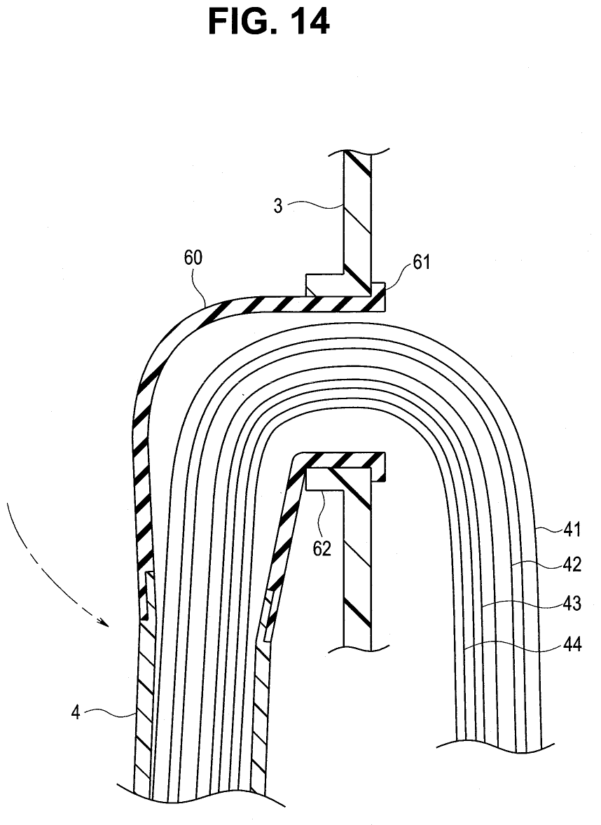

As shown in FIG. 13 and FIG. 14, in the endoscope 1, a configuration of a cable connector (first body) 60 may be a tube body formed from a rubber-like elastic member such as silicon. Note that the universal cord 4 is connected to the cable connector 60. The connector unit can be formed by an elastic material.

The cable connector 60 is coupled to a ring-like coupling member 62 provided in an exterior portion of the operation section 3. Note that, in the cable connector 60, an outward flange 61 for slip-off prevention is formed in the operation section 3.

The long members including the electric cable 41, the light guide 42 of the optical fiber, the gas feeding tube 43, and the liquid feeding tube 44 are inserted through the cable connector 60.

In this way, the universal cord 4 extended from the operation section 3 is pushed into the operation section 3 side, whereby the cable connector 60 formed from the tube body of the elastic member is easily deformed (moved).

Consequently, in the endoscope 1, by deforming the cable connector 60 with respect to the operation section 3, which is the first portion, it is possible to bring the universal cord 4, which is the second portion, close to the operation section 3 side and compactly fold the universal cord 4 extended in the axis M direction crossing the longitudinal axis L of the operation section 3.

As a result, since the endoscope 1 in this modification can also be deformed into a compact state in which the universal cord 4 comes close to the operation section 3, it is possible to improve portability at a transportation time.

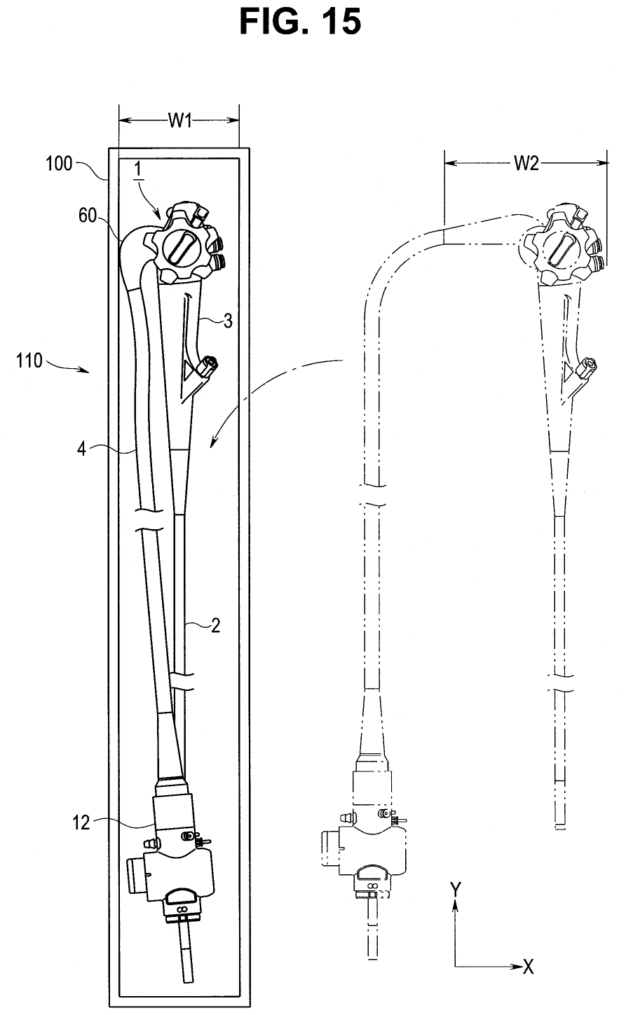

Note that, as shown in FIG. 15, when the endoscope 1 is packed in the elongated box 100 of the endoscope transportation unit 110, the cable connector 60 is pushed by an inner wall of the box 100 and deformed. Therefore, a width (distance) W1 between two inner walls facing each other in a direction (an X-axis direction in the figure) crossing a longitudinal direction of the box 100 (a Y-axis direction in the figure) is set shorter (smaller) than combined length W2 of the operation section 3 and the cable connector 60 in the direction (the X-axis direction in the figure) crossing the longitudinal direction of the endoscope 1 (the Y-axis direction in the figure) in a state in which the endoscope 1 is not stored in the box 100 (W1<W2). In particular, rigidity of a portion of the elongated box 100 in contact with the cable connector 60 can be larger than rigidity of the cable connector 60. At least a part of the case 100 has a first rigidity, the connector unit including the first body 60 has a second rigidity. The first rigidity is larger than the second rigidity.

In the embodiment and the modifications explained above, the operation section 3 and the universal cord 4 are explained as being turnably coupled by the cable connector. However, the configuration is not limited to this, and the operation section 3 and the insertion section 2 may be turnably coupled by the cable connector.

The disclosure described in the embodiment and the modifications explained above is not limited to the embodiment and the modifications. Besides, at an implementation stage, various modifications can be implemented in a range not departing from the gist of the disclosure. Further, disclosures at various stages are included in the embodiment and the modifications. Various disclosures can be extracted by appropriate combinations in a disclosed plurality of constituent elements.

For example, when the described problems can be solved and the described effects can be obtained even if several constituent elements are deleted from all the constituent elements described in the embodiment and the modifications, a configuration from which the constituent elements are deleted can be extracted as an disclosure.

### (Note 1)

An endoscope including:
a cord, through an inside of which a cable is inserted;
an operation section, through an inside of which the cable is inserted; and
a connector capable of holding the cord and the operation section in a first state and a second state, wherein
the connector is deformable such that an angle formed by the operation section and the cord is different in the first state and the second state.

### (Note 2)

An endoscope including:
a first portion, through an inside of which a cable is inserted;
a second portion, through an inside of which the cable is inserted; and
a ball joint that turnably hold the first portion and the second portion to come close to each other.

### (Note 3)

An endoscope transportation unit including:
the endoscope described in the note 1; and
a case that stores the endoscope, wherein
rigidity of at least a part of the case is larger than rigidity of a connector section of the endoscope.

### (Note 4)

The endoscope transportation unit described in the note 3, wherein a width between two inner walls facing each other in a direction crossing a longitudinal direction of the case is smaller than combined length of the operation section and the connector in a direction crossing a longitudinal direction of the endoscope in a state in which the endoscope is not stored in the case.

Example 1. An endoscope including:
a cable, through an inside of which a long member is inserted;
an operation section to which the cable is connected, the long member being inserted through an inside of the operation section; and
a connector that connects the cable and the operation section and is movably disposed to bring the cable close to the operation section.
   Example 2. The endoscope according to Example 1, wherein the connector includes a connection member that is turnable to bring the cable and the operation section close to each other.
   Example 3. The endoscope according to Example 2, wherein the connector includes a ball joint.
   Example 4. The endoscope according to Example 3, wherein
a ball of the ball joint includes a first through-hole,
a socket of the ball joint includes a second through-hole, and
the cable is inserted through the first through-hole and the second through-hole.
   Example 5. The endoscope according to Example 1, wherein the long member includes at least one of an electric cable, an optical fiber, a gas feeding tube, and a liquid feeding tube.
   Example 6. The endoscope according to Example 1, wherein
the connector includes a first tube body held to turn by the operation section and extending in a rotation axis direction, and the first tube body includes:
a first cutout formed in a circular shape on a surface of the first tube body;
a second cutout formed in a circular shape on the surface of the first tube body; and
a third cutout, a width of which in the rotation axis direction is smaller than a diameter of the first cutout, the third cutout being formed to connect the first cutout and the second cutout.
   Example 7. The endoscope according to Example 1, wherein the connector is formed by an elastic member.
   Example 8. The endoscope according to Example 1, further comprising a stopper provided in at least one of the cable, the operation section, and the connector, wherein
the stopper is configured to define a range in which the connector turns.
   Example 9. The endoscope according to Example 8, wherein
the connector includes a first tube body held to turn by the operation section and extending in a rotation axis direction and a second tube body provided on an inner circumference side of the first tube body, and
the stopper is a protrusion provided on an outer circumferential surface of the second tube body.
   Example 10. The endoscope according to Example 8, wherein
the connector includes a first tube body held to turn by the operation section and extending in a rotation axis direction and a second tube body provided on an inner circumference side of the first tube body, and
the stopper is silicone resin provided on an outer circumferential surface of the second tube body.
   Example 11. An endoscope transportation unit comprising:
an endoscope including:
   a cable, through an inside of which a long member is inserted;
   an operation section to which the cable is connected, the long member being inserted through an inside of the operation section; and
   a connector that connects the cable and the operation section and is movably disposed to bring the cable close to the operation section; and
a case that stores the endoscope, wherein
rigidity of at least a part of the case is larger than rigidity of a connector section of the endoscope.
   Example 12. The endoscope transportation unit according to Example 11, wherein a width between two inner walls facing each other in a direction crossing a longitudinal direction of the case is smaller than a combined length of the operation section and the connector in a direction crossing a longitudinal direction of the endoscope in a state in which the endoscope is not stored in the case.

What is claimed is:

1. An endoscope, comprising:
a tube including a cable;
an operation section connected to the tube, wherein the operation section inserts the cable; and
a connector unit that connects the tube and the operation section,
wherein the connector unit is movable between a first state and a second state to change a separation distance between a distal end of the tube and the operation section.

2. The endoscope according to claim 1, wherein the connector unit rotates around a connector axis,
wherein the cable extends in a first direction, and
wherein the connector axis is oriented at an angle to the first direction.

3. The endoscope according to claim 2, wherein the connector unit includes a ball joint.

4. The endoscope according to claim 3, wherein the ball joint includes a first through-hole, and
wherein the cable is inserted through the first through-hole.

5. The endoscope according to claim 4, wherein the operation section includes a second through-hole,
wherein the cable is inserted through the second through-hole, and
wherein a surface of the ball joint engages with a surface of the second through-hole so that the ball joint rotates relative to the second through-hole when the connector unit moves between the first state and the second state.

6. The endoscope according to claim 2, wherein the connector unit includes:
a first body,
a first cutout region formed in the first body, and
a second cutout region formed in the first body.

7. The endoscope according to claim 6, wherein the connector unit further includes:
a third cutout region formed in the first body and connecting the first cutout region and the second cutout region,
wherein a width of the third cutout region in a direction of the connector axis is smaller than a width of the first cutout region in the direction of the connector axis.

8. The endoscope according to claim 6, wherein the connector unit further includes a second body inserted into the first body,
wherein the first body rotates relative to the second body,
wherein the second body includes a projection on an outer circumferential surface of the second body, and
wherein the projection restricts rotation of the first body relative to the second body.

9. The endoscope according to claim 8, wherein the projection extends through the first cutout region, and
wherein, when the first body rotates relative to the second body, the projection contacts a periphery surface of the first cutout region to restrict rotation of the first body relative to the second body.

10. The endoscope according to claim 9, wherein the first body further includes a groove formed in the first body,
wherein the projection extends through the groove, and
wherein, when the first body rotates relative to the second body, the projection contacts a periphery surface of the groove to restrict rotation of the second body relative to the first body.

11. The endoscope according to claim 8, wherein the projection is formed of silicone resin.

12. The endoscope according to claim 1, wherein the cable is at least one of an electric cable, an optical fiber, a gas feeding tube, and a liquid feeding tube.

13. The endoscope according to claim 1, wherein the connector unit is formed by an elastic material.

14. The endoscope according to claim 1, wherein in the first state, the tube is located at a first angle with respect to a longitudinal axis of the operation section,
wherein, in the second state, the tube is located at a second angle with respect to the longitudinal axis of the operation section, and
wherein the first angle is larger than the second angle.

15. An endoscope transportation unit, comprising:
the endoscope according to claim 14; and
a case that stores the endoscope,
wherein, when the endoscope is in the second state, a width of the endoscope is less than a width of the case.

16. The endoscope according to claim 1, wherein, in the first state, the separation distance is a first separation distance,
wherein, in the second state, the separation distance is a second separation distance, and
wherein the first separate distance is larger than the second separation distance.

17. An endoscope transportation unit, comprising:
the endoscope according to claim 16; and
a case that stores the endoscope,
wherein, when the endoscope is in the second state, a width of the endoscope is less than a width of the case.

18. An endoscope transportation unit, comprising:
the endoscope according to claim 1; and
a case that stores the endoscope.

19. The endoscope transportation unit according to claim 18, wherein at least a part of the case has a first rigidity,
wherein the connector unit including a first body has a second rigidity, and
wherein the first rigidity is larger than the second rigidity.

20. The endoscope transportation unit according to claim 18, wherein, when the separation distance is a minimum, the separation distance is less than a width of the case.
